(19) United States
(12) Patent Application Publication
(10) Pub. No.: US 2024/0252134 A1
(43) Pub. Date: Aug. 1, 2024

(54) RADIOGRAPHY APPARATUS, CONTROL METHOD THEREFOR, AND STORAGE MEDIUM

(71) Applicant: CANON KABUSHIKI KAISHA, Tokyo (JP)

(72) Inventor: Takuya TSUKADA, Chiba (JP)

(21) Appl. No.: 18/417,007

(22) Filed: Jan. 19, 2024

(30) Foreign Application Priority Data
Jan. 31, 2023 (JP) 2023-013288

(51) Int. Cl. A61B 6/00; G16H 30/40

(52) U.S. Cl. CPC A61B 6/5294; A61B 6/54; G16H 30/40

(57) ABSTRACT

A radiography apparatus includes a photographing unit configured to acquire a radiographic image by photographing a subject, an image processing unit configured to perform image processing on the radiographic image, and an output unit configured to output a radiographic image having been subjected to the image processing and image processing information regarding the image processing in association with each other.

### BACKGROUND OF THE INVENTION

### Field of the Invention

The present invention relates to a radiography apparatus, a control method therefor, and a storage medium.

### Description of the Related Art

In hospitals, various systems cooperate to perform tests. For example, when a medical doctor judges the need for radiography of a subject, a test instruction is input from a hospital information system (HIS) terminal, and a test order is transmitted to a radiology department. The radiology department executes radiography in accordance with the test order. A radiographic image obtained by the radiography is transmitted to a picture archiving and communication systems (PACS) or printed. The radiographic system described in Japanese Patent Laid-Open No. 2017-140194 adds a photographic condition to a radiographic image. Due to this, when the radiographic image is used afterward, it is possible to recognize under what photographic condition the radiographic image is photographed. There is a case where image processing is performed on the radiographic image by a radiography apparatus after photographing. In the information added to the radiographic image by the radiography apparatus described in the above document, it is not possible to grasp the content of the image processing performed on the radiographic image.

### SUMMARY OF THE INVENTION

An aspect of the present disclosure provides a technique for making it possible to grasp the content of image processing performed on a radiographic image when the radiographic image is used afterward. According to some embodiments, a radiography apparatus comprising: a photographing unit configured to acquire a radiographic image by photographing a subject; an image processing unit configured to perform image processing on the radiographic image; and an output unit configured to output a radiographic image having been subjected to the image processing and image processing information regarding the image processing in association with each other is provided.

Further features of the present invention will become apparent from the following description of exemplary embodiments with reference to the attached drawings.

### BRIEF DESCRIPTION OF THE DRAWINGS

FIG. 1 is a block diagram illustrating an example of a radiography system according to a first embodiment.

FIG. 2 is a block diagram illustrating a functional configuration example of a control unit according to the first embodiment.

FIG. 3 is a schematic diagram illustrating a screen example for performing image processing according to the first embodiment.

FIG. 4 is a flowchart showing an operation example of the radiography apparatus according to the first embodiment.

FIG. 5 is a schematic diagram illustrating a screen example for performing image processing according to a second embodiment.

FIG. 6 is a schematic diagram illustrating a screen example for selecting a test according to the second embodiment.

FIG. 7 is a flowchart showing an operation example of the radiography apparatus according to the second embodiment.

### DESCRIPTION OF THE EMBODIMENTS

Hereinafter, embodiments will be described in detail with reference to the attached drawings. Note, the following embodiments are not intended to limit the scope of the claimed invention. Multiple features are described in the embodiments, but limitation is not made to an invention that requires all such features, and multiple such features may be combined as appropriate. Furthermore, in the attached drawings, the same reference numerals are given to the same or similar configurations, and redundant description thereof is omitted.

### First Embodiment

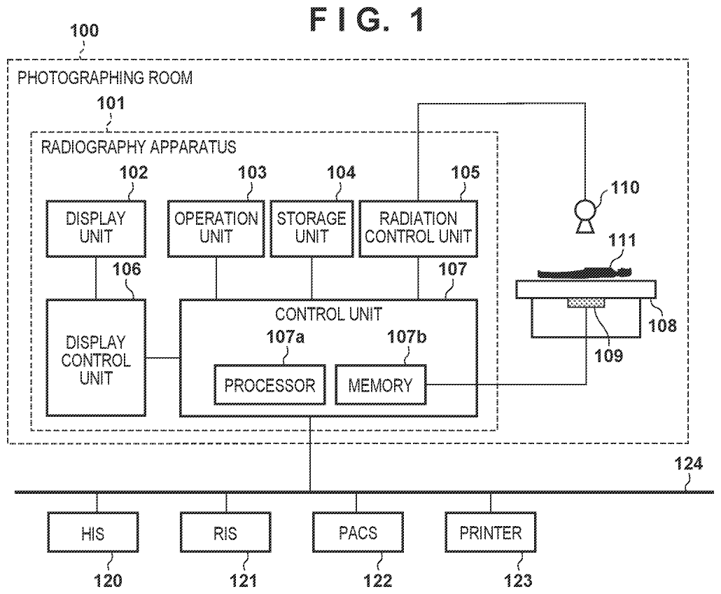

A radiography system according to the first embodiment will be described with reference to FIG. 1. As illustrated in FIG. 1, the radiography system of the present embodiment may include a radiography apparatus 101, a hospital information system (HIS) 120, a radiology information system (RIS) 121, a picture archiving and communication systems (PACS) 122, and a printer 123. The radiography system needs not include some of these components or may include other components.

The HIS 120 is a system for managing the progress of a test. The HIS 120 may include a server that manages accounting information. When performing radiography, the photographer inputs a test instruction using a terminal (input unit) of the HIS 120. In response to this input, the HIS 120 transmits request information to the radiology department of the hospital to which the radiography is requested. This request information may be called a test order. The test order includes a department name of a request source, a test ID, a test item, and patient information (subject information) regarding a subject.

The RIS 121 is a system for transmitting a test order to the radiography apparatus 101. When the RIS 121 receives a test order, the radiology department adds and transmits, to the radiography apparatus 101, photographing information (photographing region information, photographing direction information, procedure information, and the like) regarding radiography to the test order as a photographing protocol. The radiography apparatus 101 performs radiography in accordance with the received test order. The radiography apparatus 101 acquires a radiographic image having been photographed, generates test information associating the radiographic image and the test order with each other, and outputs this test information together with the radiographic image.

The PACS 122 is a system for saving and managing radiographic images. A high-definition monitor connected to the PACS 122 is used to perform radiographic image checking work, detailed postprocessing, diagnosis work, and the like. In this manner, the radiographic image acquired by the radiography apparatus 101 may be transmitted to the PACS 122. In place of this or in addition to this, the radiographic image acquired by the radiography apparatus 101 may be printed by the printer 123. Test implementation information (image ID, photographing date and time, and the like) in the radiography apparatus 101 is transmitted to the HIS 120. The implementation information transmitted to the HIS 120 may be used for accounting processing after the test in addition to the progress management of the test.

The radiography apparatus 101, the HIS 120, the RIS 121, the PACS 122, and the printer 123 are connected via a network 124 including, for example, a local area network (LAN) or a wide area network (WAN).

Each of the HIS 120, the RIS 121, and the PACS 122 may include one or more computers. The computer may include, for example, a processor such as a CPU and a memory such as a read only memory (ROM) and a random access memory (RAM). The computer may include a communication interface such as a network card and input/output devices such as a keyboard, a display, and a touchscreen. These components may be electrically connected by a bus or the like, and may be controlled by a processor executing a program stored in a memory.

As illustrated in FIG. 1, the radiography apparatus 101 that performs radiography is installed in a photographing room 100. In the photographing room 100, a radiation detector 109 that generates a radiographic image by detecting the radiation having passed through a subject 111, and a photographing table 108 are installed.

The radiography apparatus 101 includes a display unit 102 that displays a radiographic image and various types of information, an operation unit 103 operated by an operator, and a radiation control unit 105 that controls a radiation generation unit 110 that generates radiation. The radiography apparatus 101 further includes a display control unit 106 that controls the display unit 102 and a control unit 107 that controls each component. The control unit 107 may include a processor 107a and a memory 107b. The operation by the control unit 107 may be performed by the processor 107a executing a program read into the memory 107b. In place of this, at least a part of the operation of the control unit 107 may be performed by a dedicated integrated circuit such as an application specific integrated circuit (ASIC) or a field programmable gate array (FGPA). The radiography apparatus 101 may include a storage unit 104 that stores various data such as radiographic images. The storage unit 104 may include a semi-persistent storage medium such as a hard disk drive (HDD) or a solid state drive (SSD).

The radiation control unit 105 sets radiation photographic conditions in the radiation generation unit 110 and controls the radiation generation unit 110. The radiation generation unit 110 functions as a radiation source that generates radiation. The radiation generation unit 110 is achieved by, for example, a radiation tube, and irradiates the subject 111 (e.g., a specific site of the subject) with radiation. The radiation generation unit 110 can irradiate a desired irradiation range with radiation. A diaphragm that shields radiation is installed on an irradiation surface of the radiation generation unit 110. By controlling the diaphragm that shields the radiation, the operator can adjust the irradiation range of the radiation emitted from the radiation generation unit 110.

The radiography system includes the radiation detector 109 that detects radiation emitted from the radiation generation unit 110. The radiation detector 109 detects the radiation having passed through the subject 111 and outputs image data corresponding to the radiation. Note that the image data may be paraphrased as a radiographic image. Specifically, the radiation detector 109 detects the radiation having passed through the subject 111 as an electric charge corresponding to the passed radiation amount. For example, as the radiation detector 109, a direct conversion sensor that directly converts, into an electric charge, radiation such as a-Se that converts radiation into an electric charge, or an indirect sensor using a scintillator such as CsI and a photoelectric conversion element such as a-Si is used. By performing analog/digital (A/D) conversion on the detected electric charge, the radiation detector 109 generates and accumulates, in the storage unit 104, image data. The radiation detector 109 can add image information (image ID, photographing date and time, and transfer situation of image data) to the image data and transmit the image information together with the image data to the radiography apparatus 101.

The display unit 102 is achieved by, for example, a liquid crystal display or the like, and displays various types of information to the operator (e.g., a photographer, a medical doctor, or the like). The operation unit 103 includes, for example, a mouse and an operation button, and inputs various instructions from the operator to each component. The display unit 102 and the operation unit 103 may be combined to provide a graphical user interface (GUI). Note that the display unit 102 and the operation unit 103 may be achieved as a touchscreen in which they are integrated.

The control unit 107 of the radiography apparatus 101 is connected to the radiation detector 109 via a wireless LAN. Image data, a control signal, and the like are transmitted and received between the control unit 107 and the radiation detector 109. That is, the image data stored in the radiation detector 109 by radiography is output (transmitted) to the control unit 107 via the wireless LAN.

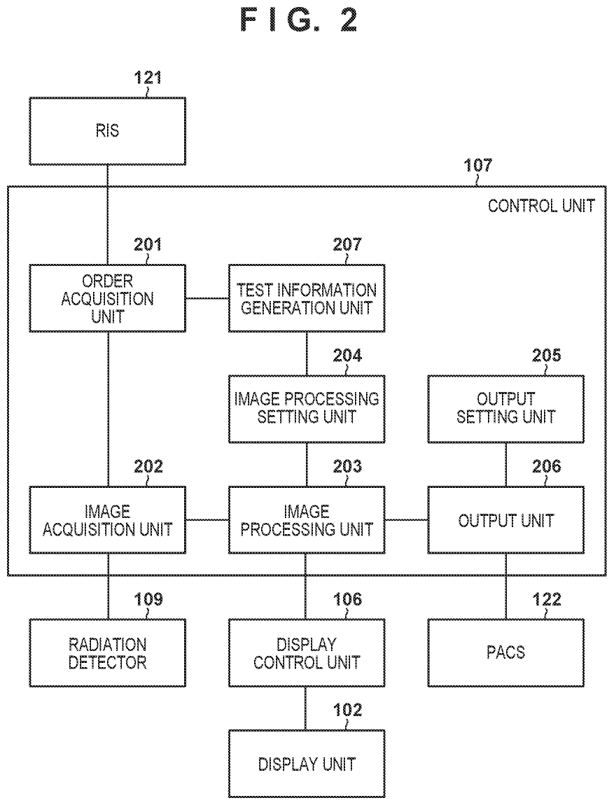

Next, a functional configuration example of the control unit 107 will be described with reference to FIG. 2. As described above, these functions may be achieved by the processor 107a executing a program stored in the memory 107b. The control unit 107 may have the components illustrated in FIG. 2. The control unit 107 need not include some of these components or may include other components.

An order acquisition unit 201 acquires, from the RIS 121, the test order to which the photographing protocol is added. By controlling the radiation control unit 105 in accordance with the photographic condition included in the test order, an image acquisition unit 202 irradiates the subject with radiation and acquires the radiographic image (image data) output from the radiation detector 109. An image processing unit 203 performs image processing on the radiographic image acquired by the image acquisition unit 202. An image processing setting unit 204 sets the content of the image processing performed by the image processing unit 203. A test information generation unit 207 generates test information associated with a radiographic image based on the test order and the setting content of the image processing. An output unit 206 outputs the radiographic image having been subjected to the image processing by the image processing unit 203 and the test information in association with each other. The output destination of the radiographic image and the test information may be the PACS 122 or may be the storage unit 104. An output setting unit 205 sets an output in the output unit 206.

The image processing unit 203 causes the display unit 102 to display, via the display control unit 106, the radiographic image acquired by the image acquisition unit 202. The display unit 102 displays this radiographic image. The display unit 102 may display setting information of image processing set in the image processing setting unit 204 and setting information of output in the output setting unit 205. Therefore, the operator can grasp what image processing is performed in the image processing unit 203 and where the radiographic image is output in the output unit 206. Then, the operator may operate setting information of image processing in the image processing setting unit 204 and setting information of output in the output setting unit 205 via the operation unit 103.

The image processing unit 203 can perform image processing on the radiographic image based on the image processing information set by the image processing setting unit 204. Specifically, the image processing unit 203 can perform image processing including brightness adjustment processing, contrast adjustment processing, edge enhancement processing, noise reduction processing, grid fringe reduction processing, scattered radiation reduction processing, and sharpness adjustment processing. The image processing setting unit 204 sets each image processing parameter required by the image processing unit 203. The test information generation unit 207 includes, into test information, information regarding the image processing set by the image processing setting unit 204. The information regarding the image processing may include, for example, a type of image processing and a parameter value used in the image processing. The information regarding the image processing may be expressed by a character string. The image processing unit 203 may replace the radiographic image displayed on the display unit 102 with a radiographic image having been subjected to image processing. This enables the operator to check the result of the image processing and further change the content of the image processing as necessary.

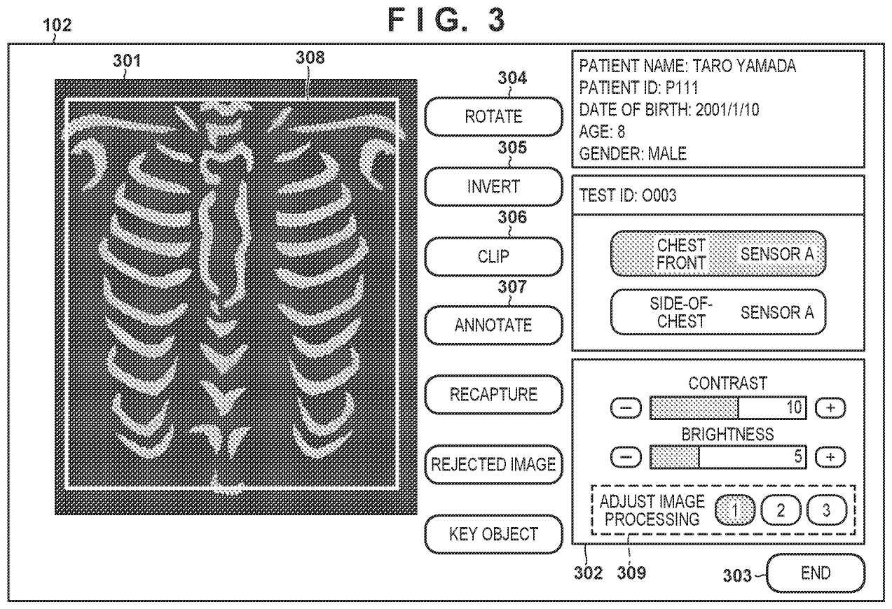

An example of a screen displayed in a GUI for performing image processing on a radiographic image will be described with reference to FIG. 3. The screen illustrated in FIG. 3 is displayed on the display unit 102. The display control unit 106 displays, in a display region 301, the radiographic image acquired by the image acquisition unit 202. The operator inputs the content (e.g., type and parameter value) of the image processing to be performed on the radiographic image displayed in the display region 301. Based on this input, the image processing setting unit 204 sets the content of the image processing to be executed. The image processing unit 203 performs the image processing having the set content on the radiographic image, and stores a new radiographic image obtained by the image processing in the memory 107b or the storage unit 104. The image processing unit 203 displays this new radiographic image in the display region of the display unit 102.

For example, when changing the appearance of the radiographic image, the operator operates a button provided in a setting region 302. When changing a display orientation of the displayed radiographic image, the operator instructs geometric transformation by operating (e.g., pressing) a rotation button 304, an inversion button 305, and the like. When changing a clipping region of the radiographic image, the operator can set the clipping region by operating a clipping button 306 and a clipping frame 308. When giving a character string to be diagnostic information to a radiographic image, the operator can operate an annotation button 307 to superimpose a graphics object, a character string, or the like on the image.

The operator can apply more advanced image processing setting by operating a button in an image processing adjustment region 309 of the setting region 302. For example, the setting region 302 may display buttons that enable setting of image processing such as edge enhancement processing and noise reduction processing in addition to brightness adjustment and contrast adjustment. These advanced image processing may be displayed by operating the button in the image processing adjustment region 309.

The operator performs the operation using the buttons as described above, and operates an end button 303 upon obtaining a desired radiographic image. Due to this, the image processing operation on the radiographic image is completed.

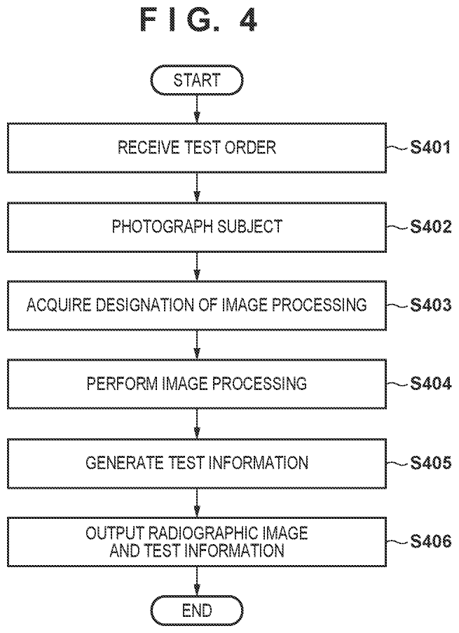

An operation example of the radiography apparatus 101 will be described with reference to FIG. 4. Each process of FIG. 4 may be performed by the processor 107a executing a program read into the memory 107b. In place of this, some or all of the processes of the operation of FIG. 4 may be executed by a dedicated processing circuit such as an ASIC or an FPGA.

In S401, the order acquisition unit 201 receives a test order from the RIS 121. The test order may include photographing information (photographing region information, photographing direction information, procedure information, and the like) as a photographing protocol. The order acquisition unit 201 may receive the test order from an apparatus different from the RIS 121. The order acquisition unit 201 stores the received test order in the memory 107b or the storage unit 104 for subsequent processing.

In S402, the image acquisition unit 202 acquires a radiographic image by photographing the subject 111 in accordance with the test order received in S401. The image acquisition unit 202 stores the acquired radiographic image in the memory 107b or the storage unit 104 for subsequent processing.

In S403, the image processing setting unit 204 acquires an instruction of image processing from the operator of the radiography apparatus 101. For example, as described with reference to FIG. 3, the image processing setting unit 204 displays, on the display unit 102, a screen including a radiographic image and a graphical object for receiving an operation. The radiographic image displayed on the display unit 102 may be an image photographed at any time by the radiography apparatus 101. For example, the image processing setting unit 204 may display a radiographic image acquired during a test of the subject 111 during the test. In place of this, the radiographic image acquired during the test of the subject 111 may be displayed after the test.

In S404, the image processing unit 203 performs image processing on the radiographic image acquired in S402 in accordance with an instruction of image processing from the operator. The image acquisition unit 202 stores the radiographic image after the image processing in the memory 107b or the storage unit 104 for subsequent processing. By repeating S403 and S404, the radiography apparatus 101 may repeatedly perform image processing on the radiographic image acquired in S402.

When the image processing in accordance with the instruction from the operator ends in S405, the test information generation unit 207 generates test information. For example, the test information generation unit 207 may include, in the test information, image processing information regarding image processing performed on the radiographic image. The image processing information may be expressed by a character string.

The test information generation unit 207 may include, in the test information, a type of the image processing and a parameter value used in the image processing. The type of the image processing and the parameter value used in the image processing may be expressed by a character string. The character string may be generated in accordance with a preset rule. For example, that the type of the image processing performed on a radiographic image is edge enhancement processing and the parameter value used in the edge enhancement processing is 10 may be expressed by a character string “E10”. In another example, that the type of the image processing performed on the radiographic image is noise reduction processing and the parameter value used in the edge enhancement processing is 5 may be expressed by a character string “N5”. That both of these processing have been performed may be expressed by a character string “E10: N5”.

The test information generation unit 207 may include, in the test information, the date when the image processing is performed. The date may be expressed by a character string such as “2023-01-23”. When image processing is executed on an identical radiographic image over a plurality of dates, the test information generation unit 207 may include any date (e.g., the latest date, all dates, or the like) in the test information.

The test information generation unit 207 may include, in the test information, information indicating a laboratory in which the image processing has been performed. This information may be expressed by a character string such as “LabA”. For each of a plurality of laboratories, an individual character string is preset by an administrator of the radiography system.

The test information generation unit 207 may include, in the test information, the photographic condition used in photographing of the subject 111. The photographic condition may be included in the test order received in S401, for example, or may be designated by the operator of the radiography apparatus 101.

In S406, the output unit 206 outputs the radiographic image having been subjected to the image processing and the test information generated in S405 in association with each other. As described above, the test information includes image processing information regarding image processing performed on the radiographic image. For example, the image processing information may be included in a test description of the test information. The image processing information may be a character string such as “LabA: 2023-01-23: E10:N5” as described above.

The output destination of the radiographic image and the test information may be the PACS 122. That is, the output unit 206 may transmit the radiographic image and the test information to the PACS 122. In place of this, the output unit 206 may transmit the radiographic image and the test information to another apparatus. The output destination of the radiographic image and the test information may be the storage unit 104. That is, the output unit 206 may store the radiographic image and the test information in the storage unit 104.

According to the above method, the image processing information regarding the image processing performed on the radiographic image is output in association with this radiographic image. Therefore, when the radiographic image is used afterward (e.g., in the PACS 122 or another apparatus), the content of the image processing performed on the radiographic image can be grasped.

### Second Embodiment

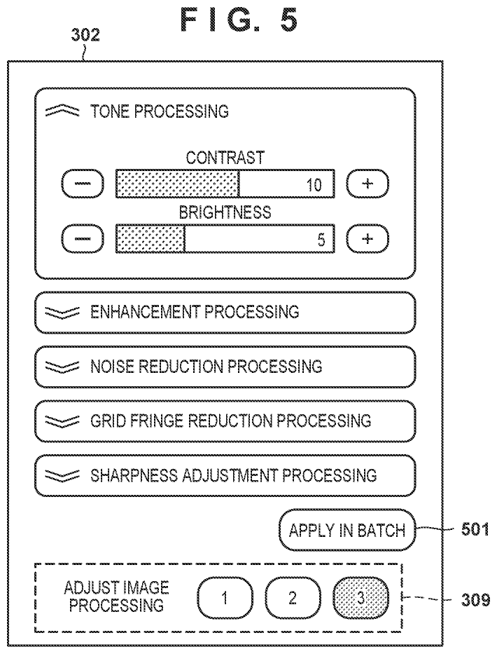

A radiography system according to the second embodiment will be described with reference to FIGS. 5 to 7. In the second embodiment, a case where image processing is performed in a batch manner on a plurality of radiographic images will be described. For matters that may be identical to those of the first embodiment, overlapping description will be omitted.

An example of a screen displayed in a GUI for performing image processing on a radiographic image will be described with reference to FIG. 5. The screen illustrated in FIG. 5 is displayed on the display unit 102. When the setting of the image processing adjustment region 309 is changed in the setting region 302 of FIG. 3, the radiography apparatus 101 displays details of the setting region 302 as illustrated in FIG. 5. This detailed screen includes a batch application button 501. By operating the batch application button 501, the operator can apply, to other radiographic images, image processing similar to the image processing applied to the radiographic image displayed in the display region 301.

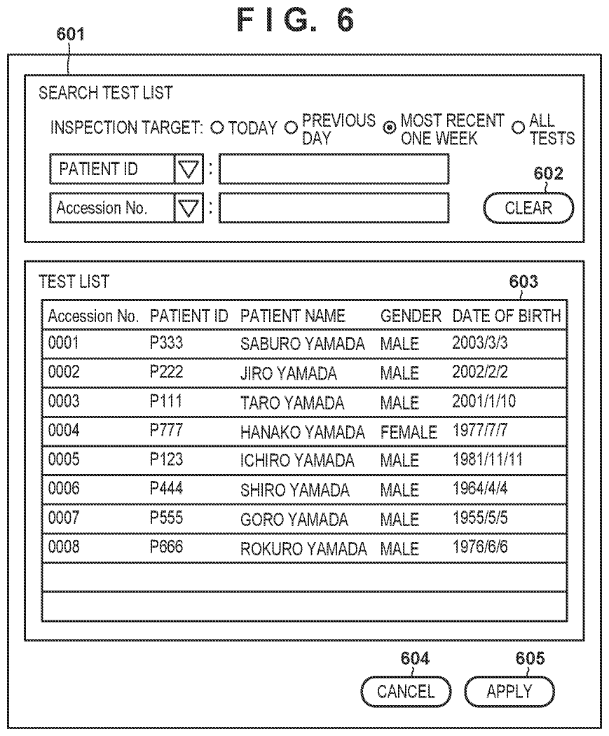

In response to the operation of the batch application button 501, the radiography apparatus 101 displays the screen as illustrated in FIG. 6. FIG. 6 is an example of a screen for selecting a test including a radiographic image to which image processing is applied.

By setting conditions in a search region 601, the operator can narrow down tests to be displayed in a list region 603. By operating a clear button 602, the operator can delete the condition having been set in the search region 601. The narrowing is released by the operation of the clear button 602, and all the tests are displayed in the list region 603.

The list region 603 displays a list of performed tests for which acquisition of the radiographic image has been completed and the test has been normally completed. The operator can select one or more tests displayed in the list region 603. When a cancel button 604 is operated, the radiography apparatus 101 closes the screen of FIG. 6 and returns to the screen of FIG. 3. In response to the operation of an application button 605 in a state where the test displayed in the list region 603 is selected, the radiography apparatus 101 applies in a batch manner the image processing to the radiographic images included in the selected test.

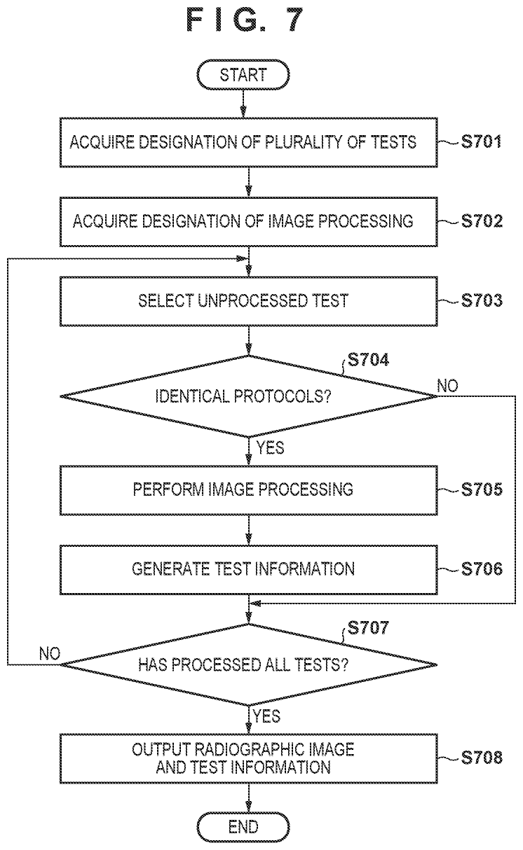

An operation example of the radiography apparatus 101 will be described with reference to FIG. 7. Each process of FIG. 7 may be performed by the processor 107a executing a program read into the memory 107b. In place of this, some or all of the processes of the operation of FIG. 7 may be executed by a dedicated processing circuit such as an ASIC or an FPGA.

In S701, the image processing setting unit 204 acquires designation of a plurality of tests from the operator. The designation of the plurality of tests may be acquired through the screen of FIG. 6, for example. Each of the plurality of tests is associated with a radiographic image generated in an individual test.

In step S702, the image processing setting unit 204 acquires, from the operator, designation of image processing to be performed on the radiographic images generated in the plurality of tests. This designation may be performed by designating a specific radiographic image subjected to image processing in the past or a test in which this radiographic image is generated. The storage unit 104 may store a radiographic image generated in the past and image processing information associated with this radiographic image.

In S703, the image processing unit 203 selects one unprocessed test from the plurality of tests designated in S701. The unprocessed test is a test for which the subsequent processing of S704 to S706 has not been performed. The test selected in S703 is represented as a target test.

In S704, the image processing unit 203 determines whether the protocol used in photographing of the subject 111 in the target test is identical to the protocol designated in S702. When determining that the protocols of the both are identical (“YES” in S704), the image processing unit 203 causes the processing to transition to S705, and otherwise (“NO” in S704), causes the processing to transition to S707. When the protocols of the both are not identical, there is a possibility that appropriate image processing cannot be performed on the radiographic image of the target test, and therefore, the subsequent processing of S705 to S706 needs not be performed.

In S705, the image processing unit 203 performs the image processing designated in S702 on the radiographic image generated in the target test. The image processing to be executed may be similar to the image processing performed in S404 of FIG. 4.

In S706, the test information generation unit 207 generates test information. The test information to be generated may be similar to the test information generated in S405 of FIG. 4. For example, the test information generation unit 207 may include, in the test information, image processing information regarding image processing performed on the radiographic image.

Furthermore, the test information generation unit 207 may include, in the test information, information indicating that image processing of the identical content has been performed on a plurality of radiographic images. For example, the test information may include a flag indicating that image processing having the identical content has been performed on a plurality of radiographic images. Furthermore, the test information may include information for specifying individual radiographic images from the plurality of radiographic images. For example, the test information generation unit 207 may generate, for each test, a character string that does not overlap among tests to which the image processing is applied in a batch manner based on the setting. The setting for generation of the character string may be performed by reading a setting file saved in the storage unit 104 of the radiography apparatus 101. For example, the information for specifying the individual radiographic images may be numbers consecutively assigned to a plurality of tests. The test information generation unit 207 may include this number in Accession No. of the test information.

In S707, the image processing unit 203 determines whether the processing of all the tests designated in S701 has ended. When determining that the processing of all the tests has ended (“YES” in S707), the image processing unit 203 causes the processing to transition to S708, and otherwise (“NO” in S707), causes the processing to transition to S703.

In S708, the output unit 206 outputs the radiographic image having been subjected to the image processing and the test information generated in S706 in association with each other for each of the plurality of radiographic images. This output may be similar to the output of S406.

According to the second embodiment, in addition to the advantages of the first embodiment, it can be grasped that image processing has been performed in a batch manner on a plurality of radiographic images. Furthermore, since individual information is added to each of the plurality of radiographic images, a specific test can be uniquely identified.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2023-013288, filed Jan. 31, 2023, which is hereby incorporated by reference herein in its entirety.

What is claimed is:

1. A radiography apparatus comprising:
a photographing unit configured to acquire a radiographic image by photographing a subject;
an image processing unit configured to perform image processing on the radiographic image; and
an output unit configured to output a radiographic image having been subjected to the image processing and image processing information regarding the image processing in association with each other.

2. The radiography apparatus according to claim 1, wherein the image processing information includes a type of the image processing and a parameter value used in the image processing.

3. The radiography apparatus according to claim 1, wherein the image processing information includes a date on which the image processing is performed.

4. The radiography apparatus according to claim 1, wherein the image processing information includes information indicating a laboratory in which the image processing has been performed.

5. The radiography apparatus according to claim 1, wherein
the image processing unit is configured to perform image processing of identical content on a plurality of radiographic images, and
the image processing information further includes information indicating that image processing of identical content has been performed on the plurality of radiographic images.

6. The radiography apparatus according to claim 5, wherein the image processing information includes information for specifying individual radiographic images from the plurality of radiographic images.

7. The radiography apparatus according to claim 1, wherein the image processing information is expressed by a character string.

8. The radiography apparatus according to claim 1, wherein the output unit is configured to output test information including the image processing information and a photographic condition used for photographing of the subject in association with a radiographic image having been subjected to the image processing.

9. A non-transitory storage medium that stores a program for causing a computer to function as the radiography apparatus described in claim 1.

10. A method for controlling a radiography apparatus, the method comprising:
acquiring a radiographic image by photographing a subject;
performing image processing on the radiographic image; and
outputting a radiographic image having been subjected to the image processing and image processing information regarding the image processing in association with each other.
